(19) United States
(12) Patent Application Publication
(10) Pub. No.: US 2021/0205494 A1
(43) Pub. Date: Jul. 8, 2021

(54) METHOD FOR PRODUCING A DENTAL IMPLANT, DENTAL IMPLANT, AND ABRASIVE BLASTING AGENT

(71) Applicant: ZV3-Zircon Vision GmbH, Wolfratshausen (DE)

(72) Inventor: Johan FEITH, Eurasburg (DE)

(21) Appl. No.: 17/059,118

(22) PCT Filed: Apr. 24, 2019
(86) PCT No.: PCT/EP2019/060518

§ 371 Date: Nov. 25, 2020

(30) Foreign Application Priority Data
May 30, 2018 (DE) 10 2018 112 935.1

(51) Int. Cl. A61L 27/32; A61C 8/00

(52) U.S. Cl. CPC A61L 27/32; A61C 8/0013; A61L 2430/12; A61C 2008/0046; A61L 2400/18; A61C 8/0037

(57) ABSTRACT

A method for producing a dental implant is provided. The method includes the steps of (a) providing a dental implant base body, especially an anchoring pin base body; (b) abrasive blasting of at least one surface portion, which is to be configured with a surface that promotes bone growth, and at least regional application of abrasive blasting agent to the surface portion; (c) at most partial removal of the abrasive blasting agent from the surface portion; and (d) sintering of the dental implant base body together with the abrasive blasting agent remaining on the surface portion.

### CROSS-REFERENCE TO RELATED APPLICATION

This application is a Section 371 of International Application No. PCT/EP2019/060518, filed Apr. 24, 2019, which was published in the German language on Dec. 5, 2019, under International Publication No. WO 2019/228713 A1, which claims priority under 35 U.S.C. § 119(b) to German Application No. 10 2018 112 935.1, filed May 30, 2018, the disclosures of each of which are incorporated herein by reference in their entireties.

### BACKGROUND OF THE INVENTION

The invention relates to a method for producing a dental implant according to claim 1. Furthermore, the invention relates to a dental implant produced according to the method according to the invention. Furthermore, the invention relates to an abrasive blasting agent for use in a method according to the invention.

It is known to produce dental implants, especially dental implant components, such as anchoring pins and/or abutments, from such a material which is related to optimal bone growth, i.e. the dental implant components are made of such a material that they can grow optimally into the jawbone.

It is also known to roughen the surfaces of dental implant components. Such roughened surfaces also improve osteogenesis.

It is known to perform such roughening by sandblasting. Afterwards, the surface is cleaned in a complex process so that abrasive blasting particles are largely removed from the surface that optimally promotes osteogenesis.

From the above it is clear that the prior art known so far with regard to optimized methods for producing dental implants has several disadvantages.

For example, special materials that may be expensive to produce have to be considered already during the forming of the dental implant component. Up to now, roughening surfaces has been associated with the disadvantage that complex cleaning measures become necessary.

### BRIEF SUMMARY OF THE INVENTION

From the aforementioned, it is thus the object of the present invention to provide a further developed method for the production of a dental implant which overcomes the aforementioned disadvantages of the prior art.

Furthermore, a dental implant is to be provided which is produced by means of a method according to the invention.

Furthermore, it is the object of the invention to specify a further developed abrasive blasting agent which can be used in a method for producing a dental implant.

According to the invention, this object is solved with regard to the method for producing a dental implant by claim 1.

With regard to the dental implant, this object is solved by the subject matter of claim 8.

With regard to the abrasive blasting agent for use in a method for producing a dental implant, this object is solved, according to the invention, by the subject matter of claim 10.

The invention is based on the idea of specifying a method for producing a dental implant comprising the following steps:
a) Provision of a dental implant base body, in particular an anchoring pin base body;
b) Abrasive blasting of at least one surface portion, which is to be configured with a surface that promotes bone growth, and at least regional application of abrasive blasting agent to the surface portion;
c) At most partial removal of the abrasive blasting agent from the surface portion;
d) Sintering of the dental implant base body together with the abrasive blasting agent remaining on the surface portion.

In other words, a base body of a dental implant is provided first. The base body can be the base body of an anchoring pin.

The base body can be designed as an oxide ceramic green body, for example, especially as a zirconium oxide green body. Preferably, this base body may have an oversize to compensate for shrinkage during any sintering or firing processes.

Prior to step b), at least one surface portion can be determined or specified to be formed with a surface that promotes bone growth.

Due to the method according to the invention, it is possible to form only a portion or only portions of the surface of the dental implant base body with a surface that promotes bone growth. Based on the method provided, it is not necessary to form the entire surface of the dental implant with a bone-growth promoting surface. However, the entire surface of the dental implant can be formed with a bone-growth promoting surface.

For example, the entire outer surface of the base body of the dental implant can be formed with such a bone-growth promoting surface.

To form this bone-growth promoting surface, the surface portion is blasted. Due to blasting within the terms of sandblasting, the surface portion is formed with a surface roughness that promotes bone growth, i.e. osteogenesis.

In addition to the formation of a surface roughness, an abrasive blasting agent is also applied at least in sections on the surface portion to be processed.

The abrasive blasting agent applied during the abrasive blasting of the surface portion impacts the surface portion. The impacted blasting agent remains on the surface, preferably mostly, especially preferably completely.

In step b), a surface with a roughness that promotes bone growth is produced, wherein particles of the abrasive blasting agent additionally serve as a surface that promotes bone growth.

In step c) the applied abrasive blasting agent is at most partially removed from the surface portion. Therefore, in step c) it is preferably not provided to perform specific cleaning steps to remove the abrasive blasting agent from the processed surface portion. Cleaning steps like washing steps or ultrasonic cleaning can thus be omitted at least partially. For example, a channel of an anchoring pin should still be cleaned after the blasting process. This cleaning can be done with the help of an ultrasonic bath.

Preferably, only dirt and/or dust particles are removed in step c), but not the abrasive blasting agent. Furthermore, it is possible that in step c) such a cleaning takes place so that loose abrasive blasting agent particles are removed. Loose abrasive blasting agent particles are especially those abrasive blasting agent particles that have not been in contact with the surface portion to be treated and/or with other abrasive blasting agent particles.

In step d) the dental implant base body is sintered together with the abrasive blasting agent remaining on the surface portion. The dental implant base body is sintered especially together with abrasive blasting agent particles which are already connected to the surface to be processed and/or with further abrasive blasting agent particles before sintering. This bond is created due to the abrasive blasting performed in step b).

It is possible that the dental implant is sintered/fired in step d) at a temperature of 1,300° C.-1,600° C., especially 1,400° C.-1,550° C.

In a sintering furnace, a slow temperature rise is preferably selected at first. Preferably, the temperature rise is approx. 100° C. per hour. As soon as the final temperature is reached, this temperature should be maintained for approx. 0.5-4.0 hours, in particular for 1.0-3.0 hours, especially preferably for approx. 2.0 hours. Then the temperature supply can be switched off, wherein the dental implant remains positioned in the sintering furnace until it has cooled down completely. Alternatively, the temperature in the sintering furnace can be lowered slowly.

In this step the final sintering or firing of the dental implant component takes place. A usable dental implant, especially a usable anchoring pin, can then be provided.

The material shrinkage after final sintering or after carrying out method step d) is 21%-27%, especially preferably 25%. This material shrinkage must be taken into account in method step b), since the roughness of the surface portion achieved after step b) decreases due to the material shrinkage.

In step b), the abrasive blasting agent is applied to the surface portion preferably at a pressure of 0.1 bar-10.0 bar, in particular of 1.0 bar-5.0 bar, especially preferably of 1.5 bar-2.5 bar.

Preferably, a nozzle with a nozzle cross-section of 0.6 mm-1.5 mm is used for the application of the abrasive blasting agent.

In step a), preferably such a dental implant base body is provided, which is presintered/pre-fired. In other words, step a) may include presintering/prefiring the dental implant body.

The dental implant component base body is preferably presintered/prefired at a temperature of 600° C.-1,100° C., in particular 700° C.-900° C. These are low presintering temperatures. The base body of the dental implant component is thus softer in comparison with base bodies from the prior art, so that the abrasive blasting agent or particles of the abrasive blasting agent can be applied more easily to the surface portion.

The pressure with which the abrasive blasting agent is applied to the surface portion in step b) must be selected on the basis of the dental implant base body provided. The softer the surface portion to be blasted, the lower the pressure or blasting pressure can be selected. The hardness of the surface portion is related to the selected presintering temperature. At a presintering temperature of 800° C., for example, a blasting pressure of approx. 1.0 bar must be selected. At a presintering temperature of 1,000° C., for example, a blasting pressure of approx. 2.0 bar should be selected.

The abrasive blasting agent can preferably be a calcium phosphate compound in powder form and/or a hydroxyapatite compound in powder form. It is therefore preferable to use an abrasive blasting agent comprising calcium phosphate powder and/or hydroxyapatite powder. Furthermore, it is possible that the abrasive consists of calcium phosphate powder and/or hydroxyapatite powder.

It is possible that a mask is applied to the base body of the dental implant before step b) so that the surface portions to be blasted are clearly separated from the surface portions not to be blasted.

Furthermore, it is possible to position and move a blasting nozzle during the blasting process in such a way that only the previously determined surface portions are blasted or sandblasted.

Preferably, the bone-growth-promoting surface of a dental implant is completely prepared before the final firing/sintering.

The advantage of the method according to the invention is that no complex cleaning processes are necessary, although blasting, in the sense of sandblasting, is performed on a surface.

A further, in particular secondary, aspect of the invention relates to a dental implant, in particular an anchoring pin, which is produced according to a method according to the invention.

Preferably, the dental implant has at least one surface portion that contains calcium phosphate particles and/or hydroxyapatite particles. The calcium phosphate particles and/or the hydroxyapatite particles are formed on at least one surface portion of the dental implant, which is formed as a surface portion that promotes bone growth. Preferably, a layer comprising calcium phosphate compounds and/or hydroxyapatite compounds is formed on the surface portion of the dental implant.

In addition, this surface portion may have a roughness that is also conducive to bone growth, i.e. osteogenesis. Preferably, the roughness of the surface portion has a maximum roughness depth Ra of 2 μm-40 μm, in particular 5 μm-15 μm.

According to one embodiment of the invention, it is possible that the anchoring pin and the abutment are integrally formed. This provides an aesthetically optimized dental implant component. Furthermore, the durability of such a dental implant component is increased.

In the described integral formation of an anchoring pin with the abutment, the dental implant component may have a surface that promotes bone growth over the entire surface.

Alternatively, a portion of the surface formed on the abutment may not have such a surface to promote bone growth. Rather, it is intended that a surface portion of the abutment has a relatively low roughness so that a crown portion can be easily placed or slid onto the abutment.

A further, in particular secondary, aspect of the invention relates to an abrasive blasting agent for use in a method, in particular the method according to the invention, for producing a dental implant.

According to the invention, the abrasive blasting agent is a powder comprising a calcium phosphate compound and/or a hydroxyapatite compound.

The abrasive blasting agent can therefore be Ca₁₀(PO₄)₆(OH)₂/Ca₃(PO₄)₂. It is also possible to use a pure hydroxyapatite powder: Ca₁₀(PO₄)₆(OH)₂.

In a particularly preferred embodiment of the invention, the powder comprises at least 5%, especially at least 10%, calcium phosphate and/or at least 30%, especially at least 50%, calcium phosphate compounds.

It is also possible that the powder contains only calcium phosphate compounds. Preferably, the powder contains no more than 30%, especially no more than 20%, of hydroxyapatite compounds.

In a particularly preferred embodiment of the invention, the particles have a size of 25 μm-150 μm, in particular of 50 μm-100 μm.

In particular, the particles of a powder to be used are irregularly shaped. Because of this, a dental implant can be produced which has the described particles on a surface portion, while at the same time a certain surface roughness can be adjusted.

### BRIEF DESCRIPTION OF THE DRAWING

The foregoing summary, as well as the following detailed description of the preferred invention, will be better understood when read in conjunction with the appended drawing:

FIG. 1 shows a dental implant component 10, which was produced according to a method according to the invention.

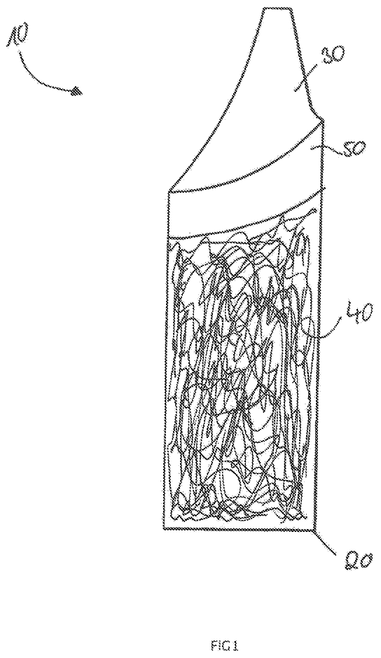

### DETAILED DESCRIPTION OF THE INVENTION

The dental implant component 10 comprises an anchoring pin 20 and an abutment 30. The anchoring pin 20 has a surface portion 40 which is formed with a surface that promotes bone growth. It can be seen that this is only a partial section of the complete surface of the anchoring pin 20. A second surface portion 50 of the anchoring pin 20, which in the implanted state lies against the gum, is not formed with a surface that promotes bone growth.

The surface portion 40 is produced in the course of a blasting process, in the sense of a sandblasting process. An abrasive blasting agent, in particular a powder comprising calcium phosphate and/or hydroxyapatite, is applied to surface portion 40.

The abrasive blasting agent is subsequently not or at least not completely removed, so that the abrasive blasting agent or the abrasive blasting agent particles represent(s) a part of the surface promoting bone growth.

Spaces may be formed between the individual particles, so that surface portion 40 also has a corresponding roughness.

In summary, a dental implant component can be made available with the aid of a simplified method, which has an improved surface, at least in portions, with regard to bone growth promotion, i.e. osteogenesis.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A method of manufacturing a dental implant (20), the method comprising by the steps of:
a) provision of a dental implant base body, especially an anchoring pin base body;
b) abrasive blasting of at least one surface portion (40), which is to be configured with a surface that promotes bone growth, and at least regional application of abrasive blasting agent to the surface portion;
c) at most partial removal of the abrasive blasting agent from the surface portion (40); and
d) sintering of the dental implant base body together with the abrasive blasting agent remaining on the surface portion (40).

2. The method according to claim 1, wherein the abrasive blasting agent is applied to the surface portion (40) at a pressure of 0.1 bar-10.0 bar.

3. The method according to claim 1, wherein step a) comprises a presintering and/or prefiring of the dental implant base body.

4. The method according to claim 3, wherein the dental implant base body is presintered and/or prefired at a temperature of 600° C.-1,100° C.

5. The method according to claim 1, wherein a calcium phosphate compound in powder form and/or a hydroxyapatite compound in powder form is used as an abrasive blasting agent.

6. The method according to claim 1, wherein the dental implant base body is sintered in step d) at a temperature of 1,300° C.-1,600° C., in particular 1,400° C.-1,550° C.

7. The method according to claim 1, wherein in step c) cleaning is carried out in such a way that loose abrasive blasting agent particles are removed.

8. A dental implant (20) produced by a method according to claim 1.

9. The dental implant (20) according to claim 8, wherein the at least one surface portion (40) comprises calcium phosphate particles and/or hydroxyapatite particles.

10. An abrasive blasting agent for use in a method according to claim 1, wherein the abrasive blasting agent is a powder comprising calcium phosphate and/or hydroxyapatite.

11. The abrasive blasting agent according to claim 10, wherein the powder comprises at least 5% calcium phosphate and/or at least 30% calcium phosphate compounds.

12. The method according to claim 2, wherein the abrasive blasting agent is applied to the surface portion (40) at a pressure of 1.0 bar-5.0 bar.

13. The method according to claim 12, wherein the abrasive blasting agent is applied to the surface portion (40) at a pressure of 1.5 bar-2.5 bar.

14. The method according to claim 4, wherein the dental implant base body is presintered and/or prefired at a temperature of 700° C.-900° C.

15. The method according to claim 6, wherein the dental implant base body is sintered in step d) at a temperature of 1,400° C.-1,550° C.

16. The abrasive blasting agent according to claim 11, wherein the powder comprises at least 10% calcium phosphate and/or at least 50% calcium phosphate compounds.
